(19) United States
(12) Patent Application Publication
(10) Pub. No.: US 2017/0273326 A1
(43) Pub. Date: Sep. 28, 2017

(54) LACTIC ACID BACTERIA-CONTAINING COMPOSITION

(71) Applicant: WAKAMOTO PHARMACEUTICAL CO., LTD., Chuo-ku, Tokyo (JP)

(72) Inventors: Kazuo TSUBOTA, Funabashi-shi (JP); Shigeru NAKAMURA, Toyooka-shi (JP); Yukari SANO, Chuo-ku (JP); Naomi GOTO, Chuo-ku (JP)

(73) Assignee: WAKAMOTO PHARMACEUTICAL CO., LTD., Chuo-ku, Tokyo (JP)

(21) Appl. No.: 15/506,940

(22) PCT Filed: Aug. 31, 2015
(86) PCT No.: PCT/JP2015/074613

§ 371 Date: Feb. 27, 2017

(30) Foreign Application Priority Data
Aug. 29, 2014 (JP) 2014-175907
May 29, 2015 (JP) 2015-110484

(51) Int. Cl. A23C 9/123; A23L 29/00; A61K 35/744; A61K 35/745

(52) U.S. Cl. CPC A23C 9/1234; A61K 35/745; A23L 29/065; A61K 35/744; A61K 35/74

(57) ABSTRACT

The present invention provides compositions incorporating lactic acid bacteria or bifidobacteria with components that act to enhance the function of lactic acid bacteria or bifidobacteria. The present invention relates to a composition containing at least one component selected from the group consisting of lutein, fish oil, lactoferrin, vitamins, γ-aminobutyric acid, and zinc, and a strain of lactic acid bacteria or bifidobacteria.

### TECHNICAL FIELD

The present invention relates to compositions containing lactic acid bacteria.

### BACKGROUND ART

Preparations of lactic acid bacteria have long been used in Japan as highly-safe drugs for controlling intestinal function. Moreover, various so-called health foods for controlling intestinal function containing lactic acid bacteria are commercially available. Furthermore, yogurt and fermented milk containing lactic acid bacteria, which have been known familiarly as healthy foods, have been approved as foods for specified health uses and attracting attention. Also in Europe and America, lactic acid bacteria-containing foods (probiotics) are gaining attention as typical foods that are not only effective in controlling intestinal function but have various other effects to contribute to maintaining health. A variety of such foods are commercially available in these countries. Lactic acid bacteria have been actively studied with the aim of developing probiotic products (Non-Patent Literature 1).

Lactic acid bacteria are known to have a variety of functions, including assistance of lactose digestion, resistance to intestinal pathogens, inhibition of colon cancer, inhibition of small intestinal bacterial overgrowth, modulation of immune functions, anti-allergic effects, reduction of blood lipid levels, antihypertensive effect, inhibition of urinary tract infection, inhibition of Helicobacter pylori infection, and inhibition of hepatic encephalopathy (Non-Patent Literature 2). It has also been reported that tooth brushing with lactic acid bacteria is effective against periodontitis (Non-Patent Literature 3). Thus, it has been revealed that lactic acid bacteria show beneficial health effects by improving flora balance not only in the intestines but also in the digestive tract, including the oral cavity or stomach, and urogenital organs such as the vagina.

Like lactic acid bacteria, bifidobacteria also have long been used as highly-safe drugs for controlling intestinal function, and various so-called health foods for controlling intestinal function containing bifidobacteria are commercially available.

Owing to increased screen time, dry air due to air conditioning, the use of contact lens, and other such factors, the number of patients with dry eye has been on the increase in recent years. Dry eye is a chronic disease that involves a decrease in tear function or keratoconjunctival epithelial disorders due to various causes, and is accompanied by ocular discomfort or visual dysfunction. Dry eye affects 10 to 20% of adults in Europe, America and Japan. The main methods conventionally employed for treating dry eye include instillation of artificial tear or synthetic compounds to supplement tear fluid or stabilize the tear film.

### CITATION LIST

### Non Patent Literature

Non-Patent Literature 1: Reuter G.: Intraintestinal Flora and Probiotics (edited by MITSUOKA Tomotari), pp. 17-39, Gakkai Shuppan Center, 1998).

Non-Patent Literature 2: Sanders ME & Huis in't Veld J: Antonie van Leeuwenhoek, 1999, vol. 76, pp. 293-315

Non-Patent Literature 3: IMAI Tatsuya: Tooth-Brushing with Lactic Acid Bacteria for Curing Periodontitis Within 3 Days, MAKINO Publishing Company, 2000

### SUMMARY OF INVENTION

### Technical Problem

Although lactic acid bacteria or bifidobacteria have a health maintaining function by themselves as mentioned above, administration of other components along with lactic acid bacteria or bifidobacteria can be expected to enhance the function of lactic acid bacteria or bifidobacteria. However, methods of combining lactic acid bacteria or bifidobacteria with other components to enhance the effects of lactic acid bacteria or bifidobacteria are yet to be enough investigated. Moreover, the effects of lactic acid bacteria or bifidobacteria in treating/preventing dry eye are yet to be enough investigated. The present invention aims to provide compositions containing lactic acid bacteria or bifidobacteria and components that enhance the functions of lactic acid bacteria or bifidobacteria.

### Solution to Problem

The present inventors have found that at least one component selected from the group consisting of lutein, fish oil, lactoferrin, vitamins, y-aminobutyric acid, and zinc enhances the function of lactic acid bacteria or bifidobacteria. The inventors have thus completed the present invention.

Specifically, the present invention relates to a composition, containing at least one component selected from the group consisting of lutein, fish oil, lactoferrin, vitamins, γ-aminobutyric acid, and zinc, and a strain of lactic acid bacteria or bifidobacteria.

The composition preferably contains lutein, fish oil, and the strain of lactic acid bacteria or bifidobacteria.

The composition preferably further contains lactoferrin.

The composition is preferably a pharmaceutical composition.

The composition is preferably a food composition.

The composition is preferably for treating or preventing dry eye.

The present invention also relates to a composition for treating or preventing dry eye, containing a microorganism of the genus Streptococcus, Enterococcus, Lactobacillus, or Bifidobacterium.

### Advantageous Effects of Invention

Since the composition of the present invention contains at least one component selected from the group consisting of lutein, fish oil, lactoferrin, vitamins, γ-aminobutyric acid, and zinc, and a strain of lactic acid bacteria or bifidobacteria, the composition is effective in enhancing the function of lactic acid bacteria or bifidobacteria.

### BRIEF DESCRIPTION OF DRAWINGS

FIG. 1 shows the results of Test Example 1.

FIG. 2 shows the results of Test Example 2.

FIG. 3 shows the results of Test Example 3.

FIG. 4 shows the results of Test Example 4.

FIG. 5 shows the results of Test Example 5.

FIG. 6 shows the results of Test Example 6.

FIG. 7 shows the results of Test Example 7.

FIG. 8 shows the results of Test Example 8.

FIG. 9 shows the results of Schirmer's test 1 in Example 2.

FIG. 10 shows the results of BUT test in Example 2.

FIG. 11 shows fluorescein staining scoring for keratoconjunctival epithelial disorders in Example 2.

FIG. 12 shows the results of DEQS in Example 2.

FIG. 13 shows the results of administration of lactic acid bacteria or bifidobacteria in Example 3.

### DESCRIPTION OF EMBODIMENTS

The present invention relates to a composition containing at least one component selected from the group consisting of lutein, fish oil, lactoferrin, vitamins, γ-aminobutyric acid, and zinc, and a strain of lactic acid bacteria or bifidobacteria.

Any lactic acid bacteria may be used as long as the effects of the present invention can be achieved. Examples include microorganisms of the genera Enterococcus, Streptococcus, Lactobacillus, Alkalibacterium, Atopobacter, Carnobacterium, Fructobacillus, Halolactibacillus, Isobaculum, Marinilactibacillus, Olsenella, Paralactobacillus, Pilibacter, Weissella, Abiotrophia, Bavariicoccus, Granulicatella, Melissococcus, Lacticigenium, Lactococcus, Leuconostoc, Oenococcus, Pediococcus, Tetragenococcus, Trichococcus, and Vagococcus.

Any microorganism of the genus Enterococcus may be used as long as the effects of the present invention can be achieved. Specific examples include Enterococcus faecium and Enterococcus faecalis. More specific examples include Enterococcus faecium WB2000 (international accession number NITE BP-01913) and Enterococcus faecium JCM5804 (available from Microbe Division, RIKEN BioResource Center).

Any microorganism of the genus Streptococcus may be used as long as the effects of the present invention can be achieved. Specific examples include Streptococcus faecalis (also called Enterococcus faecium) and Streptococcus thermophilus.

Any microorganism of the genus Lactobacillus may be used as long as the effects of the present invention can be achieved. Specific examples include Lactobaillus salivarius, Lactobacillus acidophilus, Lactobacillus casei, Lactobacillus gasseri, Lactobacillus pentosus, Lactobacillus johnsonii, Lactobacillus leuteri, Lactobacillus sanfranciscensis, Lactobacillus crispatus, Lactobacillus como, and Lactobacillus rhamnosus. More specific examples include Lactobaillus salivarius WB21 (international accession number FERM BP-7792), Lactobacillus acidophilus WB2001 (accession number NITE ABP-02109), and Lactobacillus pentosus TJ515 (accession number FERM ABP-21798). Lactobacillus acidophilus WB2001 (accession number NITE ABP-02109) was deposited at Patent Microorganisms Depositary, National Institute of Technology and Evaluation (#122, 2-5-8 Kazusa-kamatari, Kisarazu-shi, Chiba, 292-0818, Japan) on Aug. 28, 2015 under Budapest Treaty. Lactobacillus pentosus TJ515 (accession number FERM ABP-21798) was deposited at Patent Microorganisms Depositary, National Institute of Technology and Evaluation (#120, 2-5-8 Kazusa-kamatari, Kisarazu-shi, Chiba, 292-0818, Japan) on Aug. 18, 2015 under Budapest Treaty.

Microorganisms of the genus Streptococcus are preferred among them. More preferred is Streptococcus faecalis, with Streptococcus faecalis WB2000 being still more preferred.

One or two or more species of lactic acid bacteria can be used in combination. Lactic acid bacteria can be cultured by usual methods under any appropriate condition, and bacterial cells separated from the cultures by harvesting means (e.g. centrifugation) can be used in the present invention.

Lactic acid bacteria in the form of lactic acid bacterial cells, lactic acid bacteria-containing materials, culture filtrates of lactic acid bacteria, or processed products of lactic acid bacteria may be used.

Examples of the lactic acid bacterial cells include viable cells, wet cells, dried cells, and dead cells. Examples of the lactic acid bacteria-containing materials include suspensions of lactic acid bacteria, and cultures of lactic acid bacteria (each of which includes bacterial cells, a culture supernatant, and culture medium components). Examples of the culture filtrates of lactic acid bacteria include culture filtrates obtained by removing lactic acid bacterial cells from cultures of lactic acid bacteria. Examples of the processed products of lactic acid bacteria include concentrates, pastes, dried products (spray-dried products, freeze-dried products, vacuum-dried products, drum-dried products), liquids, and dilutions of lactic acid bacterial cells, lactic acid bacteria-containing materials, or culture filtrates of lactic acid bacteria.

Any amount of lactic acid bacteria may be contained. The amount is typically 0.0001 to 90% by mass, preferably 0.001 to 20% by mass, more preferably 0.01 to 10% by mass. The number of lactic acid bacteria for daily intake of the composition of the present invention is preferably 1 million to 100 billion, more preferably 10 million to 100 billion, still more preferably 100 million to 100 billion.

In the present invention, bifidobacteria can be used instead of lactic acid bacteria.

Any bifidobacteria may be used as long as the effects of the present invention can be achieved. Specific examples include microorganisms of the genus Bifidobacterium.

Any microorganism of the genus Bifidobacterium may be used as long as the effects of the present invention can be achieved. Specific examples include Bifidobacterium bifidum, Bifidobacterium longum, Bifidobacterium breve, Bifidobacterium infantis, Bifidobacterium thermophilum, Bifidobacterium pseudolongum, and Bifidobacterium pseudocatenulatum. More specific examples include Bifidobacterium longum WB1001 (accession number NITE ABP-02108). Bifidobacterium longum WB1001 (accession number MITE ABP-02108) was deposited at Patent Microorganisms Depositary, National Institute of Technology and Evaluation (#122, 2-5-8 Kazusa-kamatari, Kisarazu-shi, Chiba, 292-0818, Japan) on Aug. 28, 2015 under Budapest Treaty.

One or two or more species of bifidobacteria can be used in combination. Bifidobacteria can be cultured by usual methods at any appropriate condition, and bacterial cells separated from the cultures by harvesting means (e.g. centrifugation) can be used in the present invention.

Bifidobacteria in the form of bifidobacterial cells, bifidobacteria-containing materials, culture filtrates of bifidobacteria, or processed products of bifidobacteria may be used.

Examples of the bifidobacterial cells include viable cells, wet cells, dried cells, and dead cells. Examples of the bifidobacteria-containing materials include suspensions of bifidobacteria, and cultures of bifidobacteria (each of which includes bacterial cells, a culture supernatant, and culture medium components). Examples of the culture filtrates of bifidobacteria include culture filtrates obtained by removing bifidobacterial cells from cultures of bifidobacteria. Examples of the processed products of bifidobacteria include concentrates, pastes, dried products (spray-dried products, freeze-dried products, vacuum-dried products, drum-dried products), liquids, and dilutions of bifidobacterial cells, bifidobacteria-containing materials, or culture filtrates of bifidobacteria.

Any amount of bifidobacteria may be contained. The amount is typically 0.0001 to 90% by mass, preferably 0.001 to 20% by mass, more preferably 0.01 to 10% by mass. The number of bifidobacteria for daily intake of the composition of the present invention is preferably 1 million to 100 billion, more preferably 10 million to 100 billion, still more preferably 100 million to 100 billion.

In order to enhance the function of lactic acid bacteria or bifidobacteria, the composition contains at least one component selected from the group consisting of lutein, fish oil, lactoferrin, vitamins, γ-aminobutyric acid, and zinc, and a strain of lactic acid bacteria or bifidobacteria. The composition preferably contains lutein, fish oil, and a strain of lactic acid bacteria or bifidobacteria, and more preferably contains lutein, fish oil, lactoferrin, and a strain of lactic acid bacteria or bifidobacteria.

The amount of lutein in the composition is preferably 0.0001 to 90% by mass, more preferably 0.001 to 70% by mass, still more preferably 0.01 to 50% by mass. The lutein may be in the form of free lutein, a lutein ester, a lutein salt, or any other form. For example, marigold extract may be used as a component containing lutein.

The amount of fish oil in the composition is preferably 0.0001 to 90% by mass, more preferably 0.001 to 80% by mass, still more preferably 0.01 to 70% by mass.

The amount of lactoferrin in the composition is preferably 0.0001 to 90% by mass, more preferably 0.001 to 80% by mass, still more preferably 0.01 to 70% by mass.

Examples of vitamins include vitamin C, vitamin E, vitamin A, and vitamin B₂. Preferred among these is vitamin C or vitamin E. The amount of vitamins in the composition is preferably 0.0001 to 90% by mass, more preferably 0.001 to 70% by mass, still more preferably 0.01 to 50% by mass.

The amount of γ-aminobutyric acid in the composition is preferably 0.0001 to 90% by mass, more preferably 0.001 to 70% by mass, still more preferably 0.01 to 50% by mass. For example, rice germ extract may be used as a component containing γ-aminobutyric acid.

The amount of zinc in the composition is preferably 0.0001 to 90% by mass, more preferably 0.001 to 70% by mass, still more preferably 0.01 to 50% by mass. For example, zinc gluconate may be used as a component containing zinc.

The composition is not particularly limited as long as it can be consumed by humans or animals. The composition may be, for example, a pharmaceutical composition or a food composition.

The composition may be administered in the form of, for example, a soft capsule, a capsule, powder, fine granules, granules, a tablet, a lozenge, syrup, jelly, a suppository, cream, gel, ointment, lotion, wash, irrigation, or a liquid. These dosage forms enable safe administration or consumption.

The composition can be prepared according to usual methods using additives that can be commonly used in the field of production of pharmaceutical compositions or food compositions, such as excipients, binders, disintegrating agents, coating agents, lubricants, dispersing agents, or stabilizers.

Examples of the excipients include saccharides such as white soft sugar, lactose, mannitol, and glucose; and starches such as corn starch, potato starch, rice starch, and partly pregelatinized starch.

Examples of the binders include polysaccharides such as chitosan, dextrin, sodium alginate, carrageenan, guar gum, gum arabic, and agar; natural polymers such as tragacanth, gelatin, and gluten; cellulose derivatives such as hydroxypropylcellulose, methylcellulose, hydroxypropylmethylcellulose, ethylcellulose, hydroxypropylethylcellulose, and sodium carboxymethylcellulose; and synthetic polymers such as polyvinylpyrrolidone, polyvinyl alcohol, polyvinyl acetate, polyethylene glycol, polyacrylic acid, polymethacrylic acid, and vinyl acetate resin.

Examples of the disintegrating agents include cellulose derivatives such as carboxymethylcellulose, calcium carboxymethylcellulose, and low-substituted hydroxypropylcellulose; and starches such as sodium carboxymethyl starch, hydroxypropyl starch, corn starch, potato starch, rice starch, and partly pregelatinized starch.

Examples of the coating agents include water-insoluble polymers such as dimethylaminoethyl methacrylate/methacrylic acid copolymers, polyvinyl acetal diethylamino acetate, ethyl acrylate/methacrylic acid copolymers, ethyl acrylate/methyl methacrylate/trimethylammonium ethyl methacrylate chloride copolymers, and ethylcellulose; enteric polymers such as methacrylic acid/ethyl acrylate copolymers, hydroxypropylmethylcellulose phthalate, and hydroxypropylmethylcellulose acetate succinate; and water-soluble polymers such as methylcellulose, hydroxypropylmethylcellulose, polyvinylpyrrolidone, and polyethylene glycol.

Examples of the lubricants include talc, stearic acid, calcium stearate, magnesium stearate, colloidal silica, hydrated silicon dioxide, waxes, and hardened oil.

Examples of the dispersing agents include emulsifiers such as lecithin, glycerol fatty acid esters, and polyglycerol fatty acid esters and polysaccharide thickeners such as guar gum.

Examples of the stabilizers include beeswax, glycerol fatty acid esters, and hardened oil.

A required amount of the composition may be administered in a single dose or in multiple doses.

In the case that the composition of the present invention is a food composition, the composition may be added to food in advance or may be added to food at the time of consumption. The food may be, for example, yogurt, jelly, or modified milk. The composition may also be consumed alone as a dietary supplement or a functional food.

Since the composition of the present invention contains at least one component selected from the group consisting of lutein, fish oil, lactoferrin, vitamins, γ-aminobutyric acid, and zinc, and a strain of lactic acid bacteria or bifidobacteria, the composition can enhance the function of lactic acid bacteria or bifidobacteria. Examples of the functions of the composition of the present invention include a dry eye-treating effect, a dry eye-preventing effect, ocular infection-preventing effects, an ocular homeostasis-maintaining effect, a stress-reducing effect, an antioxidant effect, and an anti-aging effect.

Dry eye may be caused by a decrease in tear secretion in the lacrimal gland or a decrease in the amount of tear due to accelerated tear water evaporation caused by lipid or mucin abnormalities. The decrease in the amount of tear causes chronic irritation or inflammation on the corneal and conjunctival surfaces, leading to lowered quality of life of patients. Consuming the composition of the present invention can restore the tear secretion decreased by dry eye. The main methods conventionally employed for treating dry eye include instillation of artificial tear or synthetic compounds to supplement tear fluid or stabilize the tear film. The composition of the present invention, however, can be orally administered to treat or prevent dry eye. This reduces the dosing burden on patients.

In the case of the composition being used for preventing dry eye, dry eye can be prevented by long-term administration of the composition; however, single day administration can also prevent dry eye.

In the case of the composition being used for treating dry eye, dry eye can be treated by administration of the composition for one day or longer after the onset of dry eye.

### EXAMPLES

The present invention is specifically described in examples. The present invention, however, is not limited to these examples.

### Example 1

### Lactic Acid Bacteria-Containing Composition

A lactic acid bacteria-containing composition was prepared which was formed of the components shown in Table 1. The components may be conventionally known ones.

TABLE 1

| | Amount |
|---|---|
| Component | (mg/300 mg) |
| Powder of lactic acid bacteria | 5.0 |
| Fish oil | 133.5 |
| Lutein | 1.5 |
| Lactoferrin | 67.5 |
| Vitamin C | 20.0 |
| Vitamin E | 4.0 |
| Zinc | 3.52 |
| γ-aminobutyric acid | 0.25 |
| Emulsifier | 20.5 |

Test Examples 1 to 8 were performed using the following test animals, stressing method, tear secretion measurement method, and statistical analysis method.

### (Test Animal)

The test animals used were female 7 to 8 week old C57BL/6 mice acclimatized for one week in a breeding room in an environment maintained at a lighting period of 12 hours, a room temperature of 23±5° C., and a relative humidity of 60±10%.

### (Stressing Method)

Each test animal was restrained for four consecutive hours once daily in a polypropylene centrifuge tube (volume: about 60 mL) treated to allow the test animal to breathe and excrete. Air was blown (at a velocity of 0.5 to 1.0 m/S) onto the face of the restrained test animal, whereby she was subjected to stressing. When not subjected to the stressing treatment, the test animals were allowed free access to chow (solid chow, mouse/rat/hamster chow MF, produced by Oriental Yeast Co., Ltd.) and water (tap water) in the cage. Five to six mice per group were tested.

### (Tear Secretion Measurement Method)

Prior to the stressing treatment, a cotton thread (ZONE-QUICK (registered trademark), Showa Yakuhin Kako Co., Ltd.) was inserted into the lateral canthi of both eyes of each test animal for 15 seconds. The length of the portion of the cotton thread browned by penetration of tear fluid was measured with a precision of 0.5 mm. The average of both eyes of each individual was taken as the amount of tear secretion thereof.

### (Statistical Analysis Method)

Data was statistically analyzed using statistical software SAS (produced by SAS Institute Inc.) and StatLight (produced by Yukms Co., Ltd.). A t-test or Dunnett test compared with a control group and a paired t-test compared with an untreated (before treatment) group were performed. Each test was carried out at a two-sided significance level of 5%, and a P value of less than 0.05 was considered significant. The average and the standard deviation were also determined.

### Test Example 1

### Prophylactic Effect Test 1

The composition of Example 1 was orally administered to test animals once daily at a dose of 10 mg/kg or 50 mg/kg on the day before the stressing treatment and during the stressing treatment period, or at a dose of 10 mg/kg for five days before the stressing treatment and during the stressing treatment period. Test animals as a control group did not receive the composition of Example 1. These test animals were subjected to the stressing treatment for three days. The amount of tear secretion of the test animals was measured each day and statistically analyzed.

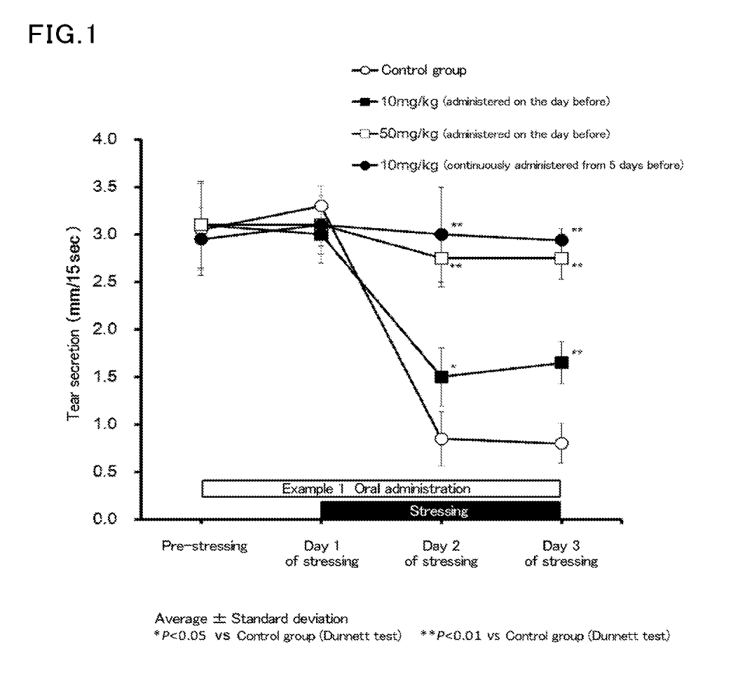

FIG. 1 shows the results. The test animals not receiving the composition of Example 1 showed a great decrease in tear secretion due to the stressing treatment. The prior administration of the composition of Example 1 prevented a decrease in tear secretion. It is demonstrated that the consumption of the composition of Example 1 at a higher dose in a short period of time (50 mg/kg on the day before) or at a low dose but for a longer period of time (10 mg/kg for five days) resulted in less decrease in tear secretion, indicating a higher dry eye-preventing effect.

### Test Example 2

### Prophylactic Effect Test 2

The composition of Example 1 was orally administered to test animals at a dose of 10 mg/kg once daily from five days before the stressing treatment to five days after the start of the stressing treatment. The test animals were subjected to the stressing treatment for seven days. The amount of tear secretion of the test animals was measured each day and statistically analyzed.

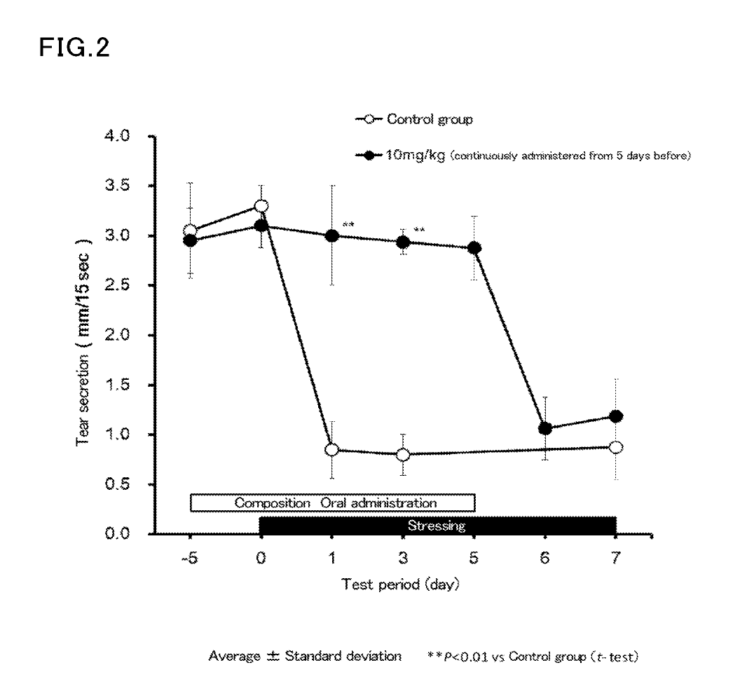

FIG. 2 shows the results. The test animals not receiving the composition of Example 1 showed a great decrease in tear secretion due to the stressing treatment. In the test animals receiving the composition of Example 1, the decrease in tear secretion was inhibited during the administration period, but the amount of tear secretion decreased when the administration was discontinued. These results demonstrated that long-term continuous consumption of the composition of Example 1 can achieve a particularly large effect in preventing dry eye.

### Test Example 3

### Prophylactic Effect Test 3

From 5 or 14 days before the stressing treatment to the end of the stressing treatment, test animals were allowed free access to chow mixed with the composition of Example 1 at a concentration of 0.06%. The test animals were subjected to the stressing treatment for five days. The amount of tear secretion was measured each day and statistically analyzed.

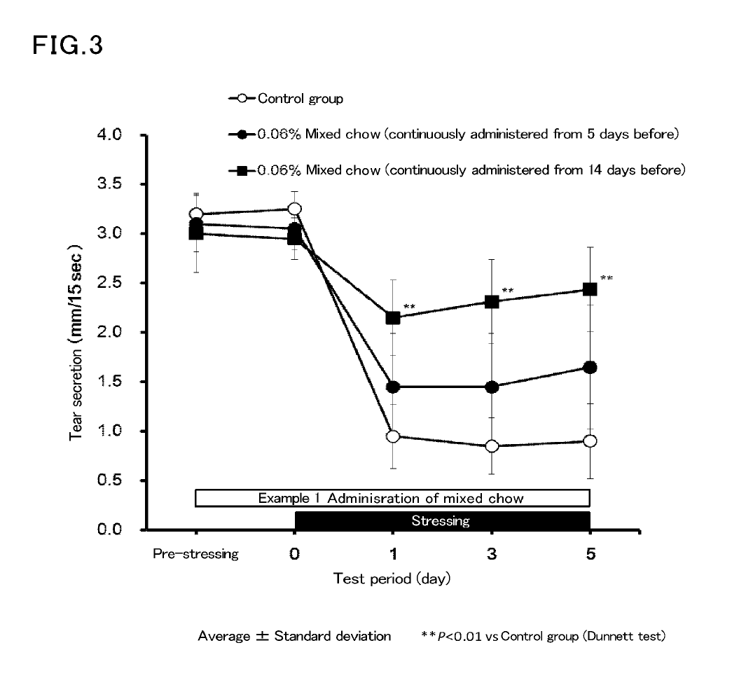

FIG. 3 shows the results. The test animals receiving chow not containing the composition of Example 1 showed a great decrease in tear secretion due to the stressing treatment.

In the test animals receiving chow mixed with the composition of Example 1, it is demonstrated that a longer administration period resulted in less decrease in tear secretion. These results demonstrated that long-term continuous consumption of the composition of Example 1 as food can achieve a particularly large effect in preventing dry eye.

### Test Example 4

### Therapeutic Effect Test 1

The composition of Example 1 was orally administered to test animals which showed a decrease in tear secretion after the stressing treatment, at a dose of 5 mg/kg, 10 mg/kg, or 50 mg/kg once daily for nine days. Test animals as a control group did not receive the composition of Example 1. The stressing treatment was performed during the period of administration of the composition of Example 1. The amount of tear secretion of the test animals was measured each day and statistically analyzed.

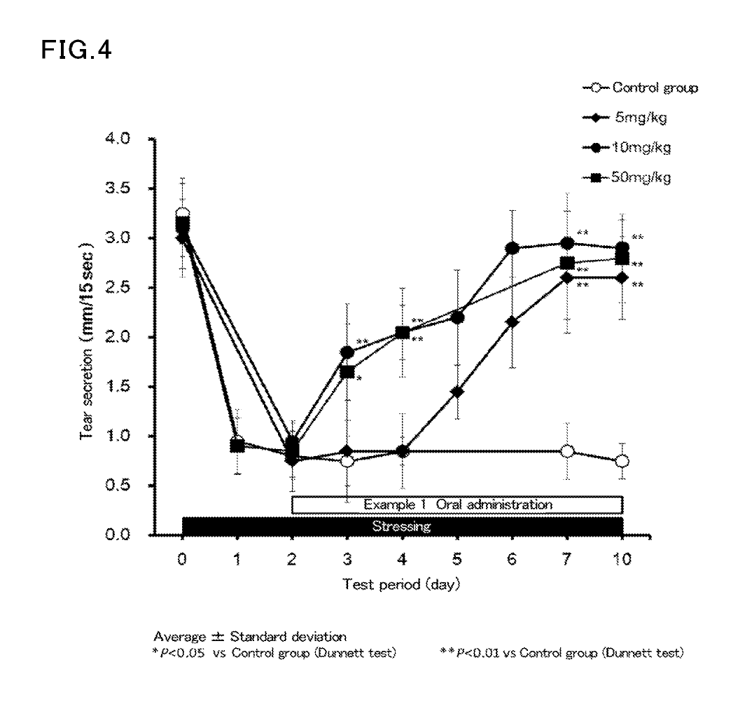

FIG. 4 shows the results. The test animals not receiving the composition of Example 1 did not show any restoration of the amount of tear secretion. The test animals receiving the composition of Example 1 showed a restoration of the amount of tear secretion depending on the dose and the administration period. These results demonstrated that consumption of the composition of Example 1 after the onset of dry eye can achieve a dry eye-treating effect.

### Test Example 5

### Therapeutic Effect Test 2

The composition of Example 1 was orally administered to test animals which showed a decrease in tear secretion after the stressing treatment, at a dose of 50 mg/kg once daily for nine days. The stressing treatment was performed during the administration period and for three days after the end of administration. The amount of tear secretion of the test animals was measured each day and statistically analyzed.

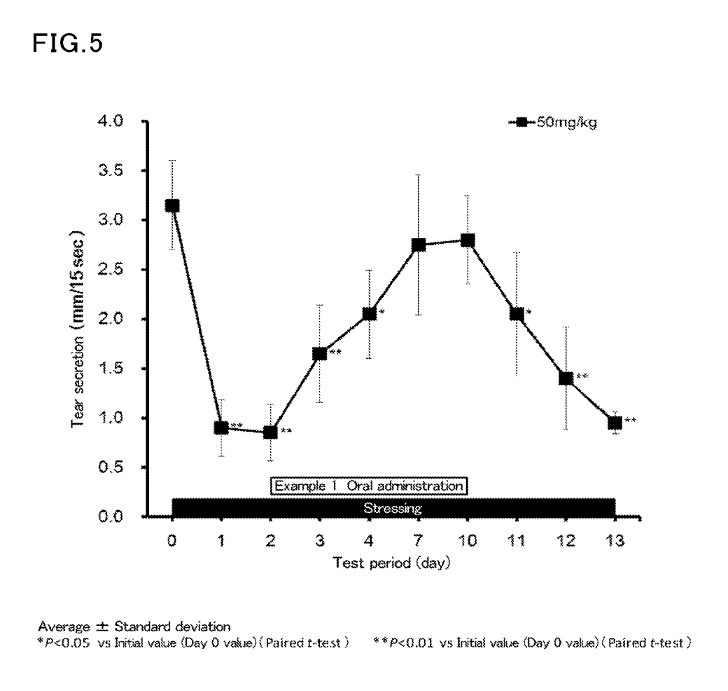

FIG. 5 shows the results. The tear secretion of the test animals that had been low due to the stressing treatment began to recover when the oral administration of the composition of Example 1 was started. The recovery progressed as the oral administration of the composition of Example 1 was continued, and the amount of tear secretion approached the value before the stressing treatment. Thereafter, when the oral administration of the composition of Example 1 was discontinued, the tear secretion of the test animals decreased again. These results demonstrated that continuous consumption of the composition of Example 1 can achieve a particularly large effect in treating dry eye.

### Comparative Example 1

In order to demonstrate the dry eye-preventing effect of lactic acid bacteria, a composition containing all the components shown in Table 1 except the lactic acid bacteria was prepared as Comparative Example 1.

### Test Example 6

### Prophylactic Effect Test 4

A single dose of the composition of Example 1 or the composition of Comparative Example 1 at 50 mg/kg was orally administered to test animals on the day before the stressing treatment. Test animals as a control group did not receive the composition of the present invention on the day before the stressing treatment. The amount of tear secretion of the test animals was measured on the day before the stressing treatment, just before the stressing treatment, and the day after the stressing treatment. The amount of tear secretion was statistically analyzed.

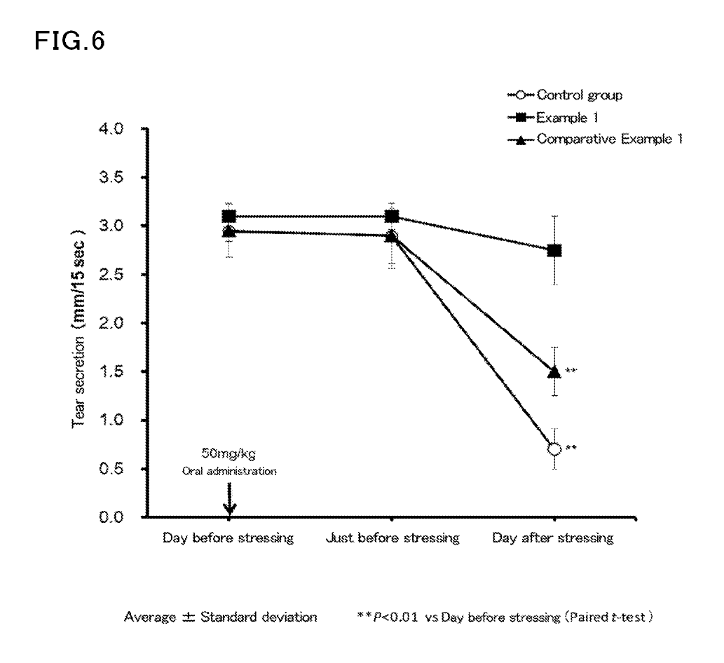

FIG. 6 shows the results. The test animals without oral administration of the composition of Example 1 showed a great decrease in tear secretion due to the stressing treatment. The test animals with oral administration of the composition of Comparative Example 1 also showed a decrease in tear secretion due to the stressing treatment. The test animals with oral administration of the composition of Example 1 showed almost no decrease in tear secretion due to the stressing treatment. These results demonstrated that it is important for the dry eye-preventing effect that the composition of Example 1 contains lactic acid bacteria.

### Test Example 7

### Therapeutic Effect Test 3

The (therapeutic) effect of restoring the amount of tear secretion after a long-term stressing test was tested. Test animals were subjected to the stressing treatment for 35 consecutive days. From Day 21 to Day 28 after the start of the stressing treatment, a single dose of the composition of Comparative Example 1 at 10 mg/kg was orally administered to the test animals. Thereafter, for one week from Day 29 to Day 36 after the start of the stressing treatment, a single dose of the composition of Example 1 at 10 mg/kg was orally administered to the test animals. The amount of tear secretion of the test animals was measured each day and statistically analyzed.

FIG. 7 shows the results. In the mice to which the composition of Comparative Example 1 was orally administered from Day 21 to Day 28 after the start of the stressing treatment, the amount of tear secretion remained low from Day 1 to Day 36 after the start of the stressing treatment, as in the control group. On the other hand, in the test animals to which the composition of Example 1 was administered from Day 29 to Day 36 after the start of the stressing treatment, the tear secretion that had been low since Day 1 after the start of the stressing treatment gradually recovered from Day 29 after the start of the stressing treatment, where the administration of the composition of Example 1 was started. On Day 35 after the start of the stressing treatment, the tear secretion recovered to exceed the amount of tear secretion before the start of the stressing treatment. These results demonstrated that it is important for the dry eye-treating effect that the composition of Example 1 contains lactic acid bacteria.

### Test Example 8

### Therapeutic Effect Test 4

The (therapeutic) effect of restoring the amount of tear secretion after a long-term stressing test was tested. Test animals were subjected to the stressing treatment for 40 consecutive days. From Day 13 to Day 21 after the start of the stressing treatment, a single dose of the composition of Example 1 at 50 mg/kg was orally administered to the test animals. After a washout period from Day 22 to Day 28 after the start of the stressing treatment, a single dose of the composition of Example 1 at 10 mg/kg was orally administered to the test animals from Day 29 to Day 40. The amount of tear secretion of the test animals was measured each day and statistically analyzed.

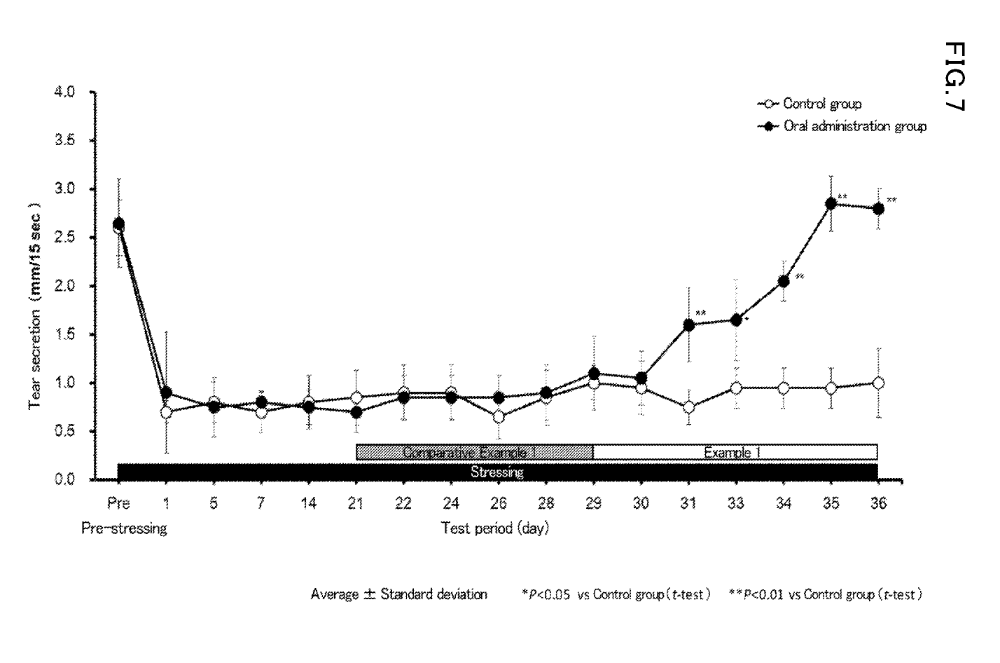

FIG. 8 shows the results. The test animals not receiving the composition of Example 1 showed a decrease in tear secretion from Day 1 after the start of the stressing treatment and then did not show any restoration of the amount of tear secretion. On the other hand, in the test animals to which a single dose of the composition of Example 1 at 50 mg/kg was orally administered, the tear secretion that had been low since Day 1 after the start of the stressing treatment gradually recovered from Day 14 to Day 21 after the start of the stressing treatment. During the washout period from Day 22 to Day 28 after the start of the stressing treatment, the tear secretion showed a tendency to decrease, but from Day 29 to Day 40, during which a single dose of the composition of Example 1 at 10 mg/kg was orally administered, the tear secretion gradually recovered. These results demonstrated that it is important for the dry eye-treating effect that the composition of Example 1 contains lactic acid bacteria.

### Example 2

### Effect of Lactic Acid Bacteria-Containing Composition on Humans

### (Lactic Acid Bacteria-Containing Composition and Administration Method Thereof)

Twenty men and women from 22 to 59 years of age with subjective dry eye symptoms were given soft capsules containing the components shown in Table 2 at a dose of two capsules once daily after dinner for eight weeks.

TABLE 2

| | Amount |
|---|---|
| Component | (mg/capsule) |
| Fish oil | 266.0 |
| Lactoferrin concentrate | 75.0 |
| Zinc gluconate | 27.5 |
| Vitamin C | 20.0 |
| Marigold extract | 7.5 |
| Vitamin E-containing vegetable oil | 6.0 |
| Powder of lactic acid bacteria | 5.0 |
| GABA-containing rice germ extract | 5.0 |
| Emulsifier | 38.0 |

### (Test Method)

A dry eye test was performed before and after the consumption of the soft capsules, i.e. twice in total. In the test, three items for ocular symptoms (Schirmer's test 1, BUT test, fluorescein staining scoring for keratoconjunctival epithelial disorders) were performed on all the eyes, and two subjective symptom questionnaires (Dry Eye-related Quality-of-life Score (DEQS) (Japan), VAS assessment of subjective ocular symptoms (11 items)) were also performed. The Schirmer's test 1, BUT test, and fluorescein staining scoring for keratoconjunctival epithelial disorders were carried out in conformity with the 2006 Diagnostic Criteria for Dry Eye (Japan). For DEQS, a questionnaire developed by the Dry Eye Society was used.

(Results and Discussion)

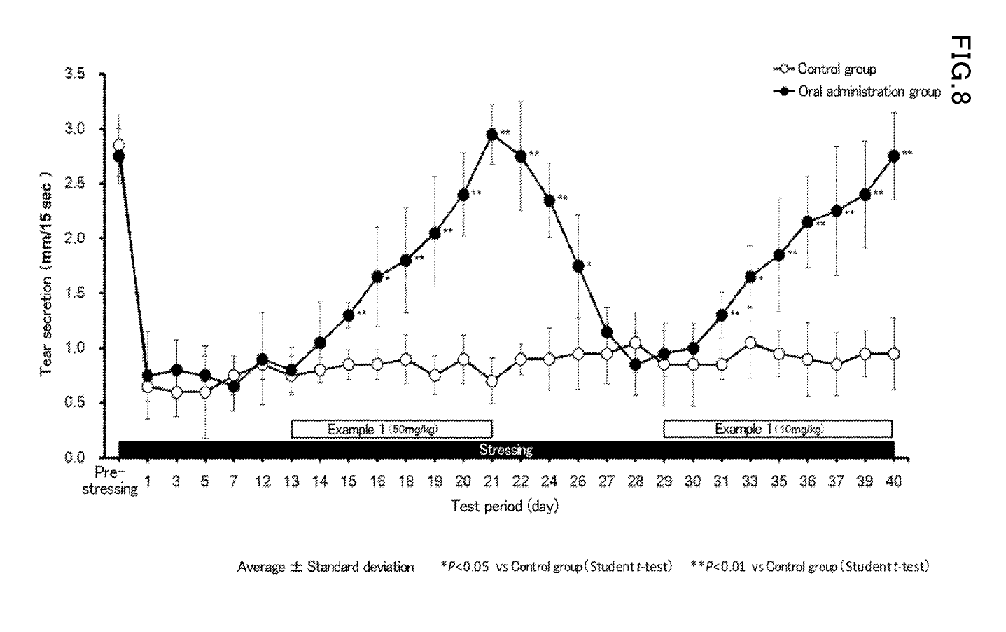

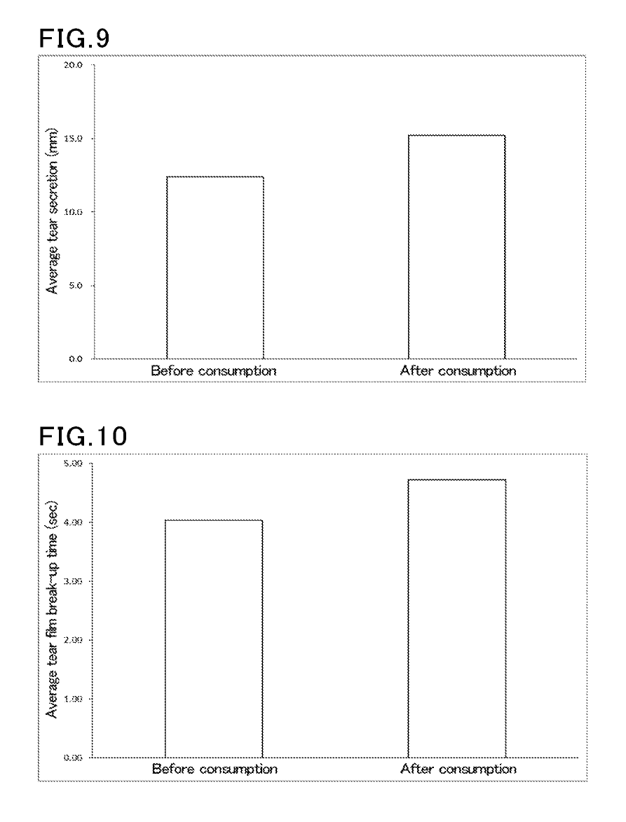

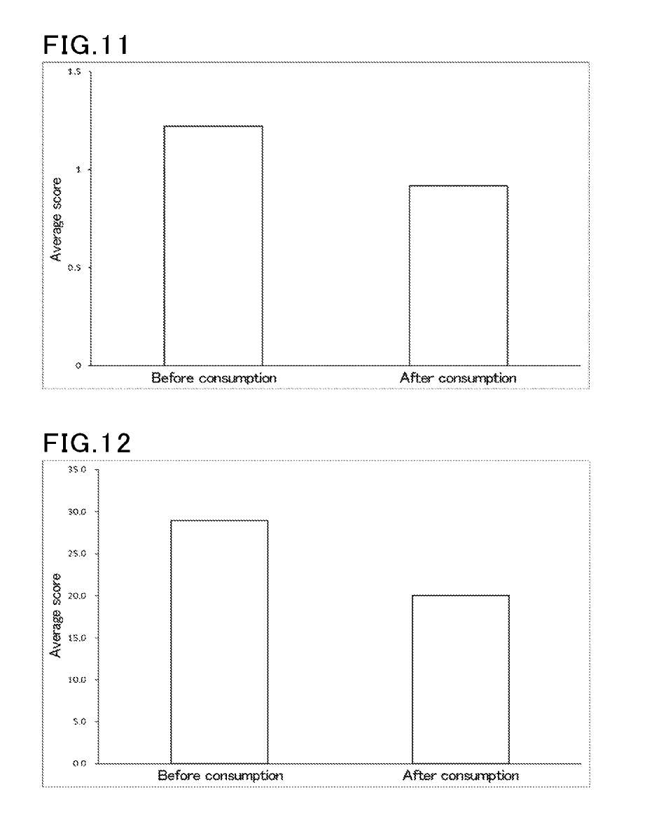

FIGS. 9 to 12 and Table 3 show the results of 18 subjects, excluding two who were unable to take some of the tests due to pain or other reasons. The results were improved on all the ocular symptom test items after the consumption of the soft capsules. Moreover, the scores of the subjective symptom questionnaires were also improved after the consumption of the soft capsules. These results suggested that the composition of the present invention is effective in improving dry eye symptoms.

TABLE 3

| | Before | After |
|---|---|---|
| Item | administration | administration |
| Dry eyes | 46.04 | 31.51 |
| Difficulty opening eyes | 17.29 | 11.87 |
| Gritty eyes | 36.14 | 21.11 |
| Eye pain | 27.37 | 20.03 |
| Red eyes | 14.33 | 13.64 |
| Mucous discharge | 26.86 | 17.08 |
| Itchy eyes | 27.38 | 14.94 |
| Blurred vision | 33.42 | 24.85 |
| Sensitivity to light | 23.03 | 15.91 |
| Heavy eyes | 24.41 | 14.38 |
| Tired eyes | 56.06 | 36.27 |

### Example 3

### Dry eye-treating or -preventing effects of lactic acid bacteria or bifidobacteria

### (Test Animal)

The test animals used were female 7 to 8 week-old C57BL/6 mice acclimatized for one week in a breeding room in an environment maintained at a lighting period of 12 hours, a room temperature of 23±5° C., and a relative humidity of 60±10%.

### (Stressing Method)

Each test animal was restrained for four consecutive hours once daily in a polypropylene centrifuge tube (volume: about 60 mL) treated to allow the test animal to breathe and excrete. Air was blown (at a velocity of 0.5 to 1.0 m/S) onto the face of the restrained test animal, whereby she was subjected to stressing. When not subjected to the stressing treatment, the test animals were allowed free access to chow (solid chow, mouse/rat/hamster chow MF, produced by Oriental Yeast Co., Ltd.) and water (tap water) in the cage. Five to six mice per group were tested.

(Tear Secretion Measurement Method)

Prior to the stressing treatment, a cotton thread (ZONE-QUICK (registered trademark), Showa Yakuhin Kako Co., Ltd.) was inserted into the lateral canthi of both eyes of each test animal for 15 seconds. The length of the portion of the cotton thread browned by penetration of tear fluid was measured with a precision of 0.5 mm. The average of both eyes of each individual was taken as the amount of tear secretion thereof.

### (Lactic Acid Bacteria and Bifidobacteria)

Freeze-dried powder of Streptococcus faecalis WB2000 (bacteriologically, Enterococcus faecium WB2000), Enterococcus faecium JCM5804, Lactobacillus salivarius WB21, Lactobacillus acidophilus WB2001, Lactobacillus pentosus TJ515, or Bifidobacterium longum WB1001 was individually suspended in 0.5 mL of distilled water such that the resulting suspension contained 0.34 mg of the powder. On the day before the stressing treatment and during the stressing treatment period, the suspension was orally administered to test animals once daily at a dose of 17 mg/kg, calculated as the freeze-dried powder of bacteria. Test animals as a control group did not receive the above lactic acid bacteria or bifidobacteria. These test animals were subjected to the stressing treatment for four days. The amount of tear secretion of the test animals was measured on the day before the stressing treatment, Day 2 of the stressing treatment, and Day 4 of the stressing treatment.

### (Results and Discussion)

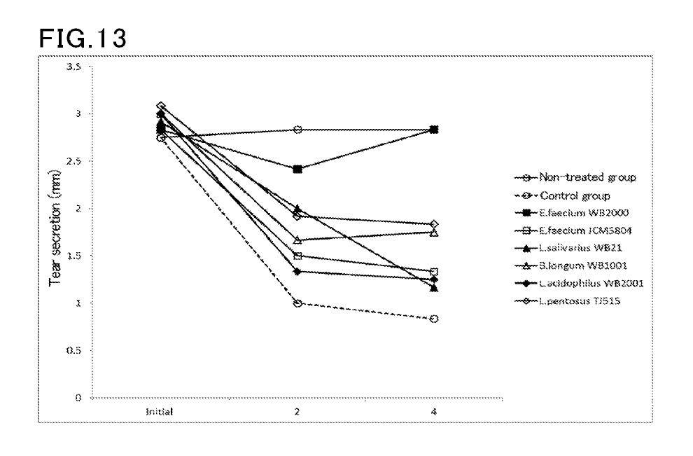

FIG. 13 shows the results. The test animals of the control group showed a great decrease in tear secretion due to the stressing treatment. The prior administration of the above lactic acid bacteria or bifidobacteria resulted in less decrease in tear secretion, indicating a dry eye-preventing effect. In particular, it is demonstrated that Streptococcus faecalis WB2000 particularly inhibited the decrease in tear secretion as compared to the other bacteria, thus exhibiting a high dry eye-preventing effect.

1. A composition, comprising:
at least one component selected from the group consisting of lutein, fish oil, lactoferrin, vitamins, γ-aminobutyric acid, and zinc; and
a strain of lactic acid bacteria or bifidobacteria.

2. The composition according to claim 1, comprising lutein, fish oil, and the strain of lactic acid bacteria or bifidobacteria.

3. The composition according to claim 2, further comprising lactoferrin.

4. The composition according to claim 1, which is a pharmaceutical composition.

5. The composition according to claim 1, which is a food composition.

6. The composition according to claim 1, which is suitable for treating or preventing dry eye.

7. A composition comprising:
a microorganism of the genus Streptococcus, Enterococcus, Lactobacillus, or Bifidobacterium,
wherein the composition is suitable for treating or preventing dry eye.

8. A method for treating dry eye, comprising administering the composition according to claim 1 to a subject in need thereof.

9. A method for treating dry eye, comprising administering the composition according to claim 7 to a subject in need thereof.
